(19) United States
(12) Patent Application Publication
(10) Pub. No.: US 2023/0233433 A1
(43) Pub. Date: Jul. 27, 2023

(54) Cosmetic composition for improving appearance of scars

(71) Applicant: Ieshia Stewart, Maple Grove, MN (US)

(72) Inventor: Ieshia Stewart, Maple Grove, MN (US)

(21) Appl. No.: 18/093,014

(22) Filed: Jan. 4, 2023

Related U.S. Application Data
(60) Provisional application No. 63/303,098, filed on Jan. 26, 2022.

(51) Int. Cl. A61K 8/44; A61K 8/67; A61K 8/66; A61K 8/35; A61K 8/9789; A61K 8/9794; A61K 8/73; A61Q 19/00; A61K 8/891

(52) U.S. Cl. CPC A61K 8/442; A61K 8/44; A61K 8/676; A61K 8/678; A61K 8/675; A61K 8/66; A61K 8/35; A61K 8/9789; A61K 8/9794; A61K 8/735; A61Q 19/00; A61K 8/891

(57) ABSTRACT

A cosmetic composition for topical skin application and for use in reducing the appearance of scars comprising a combination of arginine and glutamine. The composition comprises L-Arginine in combination for L-Glutamine and further comprises bromelain, tetrahydrobisdemethoxydiferuloylmethane and tetrahydrocurcumin. Additional ingredients include various antioxidants, moisturizers, skin protectants, and vitamins together to promote the healing of affected skin while reducing visible scarring.

### CROSS-REFERENCE TO RELATED APPLICATIONS

This application is based on and claims the benefit of U.S. provisional application Ser. No. 63/303,098, filed on Jan. 26, 2022, the content of which is incorporated herein in its entirety.

### BACKGROUND

The present invention relates to a cosmetic composition and more specifically to a composition for topical application to skin for improving the appearance of scar tissue.

Normal skin is composed of an abundance of collagen, where the collagen is arranged in a basket weave formation. Collagen is the most abundant protein in the body and can be found in the muscles, skin, and bones. The main purpose of collagen is to provide structure and hold or mend these other structures together.

When a break in the skin occurs, the body sends blood flow and fibroblast to the point of the skin breakdown. Those fibroblast in turn produce collagen. Collagen fibers migrate to the point of the skin break down as a result of a triggered immune and healing response. This collagen usually is aligned in a single direction, and this is what causes scarring.

The goal for wound healing is always to heal within, healing first the deeper inner structures and tissue to the outer superficial dermal layers. Proper wound healing can result in a scar that is less noticeable and aesthetically appealing. Within the initial phase of wound healing, scars can be red or dark and raised due to inflammation. With consistent attention and treatment, the scar may become pale or flesh tone and smoother. The inevitable reality of scarring reveals that the results remain individualized to the genetics, the origin of skin breakdown, length of time, among other variables.

Excessive scarring results when your body attempts to repair and heal a break within the dermis. When the deepest layers of skin have been punctured, the body's immune system attempts to recover by producing collagen to mend the wound, but more collagen is produced than is degraded. This imbalance is what contributes to excessive and abnormal scarring.

Protein is important in promotion of proper wound healing. Protein is needed to help build and repair muscle, skin, and other body tissues. Generally, patients are encouraged to supplement/utilize a diet with sufficient protein to aid wound healing.

### SUMMARY

An aspect of the present disclosure relates to a cosmetic composition for topical application for reducing the appearance of scars. The cosmetic composition comprises a combination of arginine and glutamine; one or more skin protectants; one or more moisturizing agents; one or more anti-inflammatories; one or more emulsifiers; one or more emollients; one or more probiotics; a natural preservative; vitamin c, vitamin B-3, vitamin E or a combination thereof; and silicone.

Another aspect of the present disclosure relates to a method of reducing the appearance of a scar on affected skin. The method comprises topically applying a cosmetic composition to affected skin, wherein the cosmetic composition comprises a combination of arginine and glutamine; one or more skin protectants; one or more anti-inflammatories; one or more moisturizing agents; one or more emulsifiers; one or more emollients; one or more probiotics; a natural preservative; vitamin c, vitamin B-3, vitamin E or a combination thereof; and silicone.

In one more embodiments described herein, the composition comprises L-Arginine in a concentration of about 0.01% by weight to about 25% by weight and L-Glutamine in a concentration of about 0.002% by weight to about 20% by weight.

In one or more embodiments, the composition further comprises bromelain, tetrahydrobisdemethoxydiferuloylmethane, and tetrahydrocurcumin.

Yet another aspect of the present disclosure relates to an active composition for use in a cosmetic composition for topical application, the active composition comprising a combination of at least two amino acids, niacinamide, bromelain, arnica, hyaluronic acid, turmeric, and vitamin E.

### DETAILED DESCRIPTION

The composition according to one or more embodiments described herein is a unique, original blend of ingredients and can be considered an all-in-one combination of ingredients that support the improvement in the appearance of scars.

The composition may be provided for topical use, including as a cream, lotion, tonic, serum, or ointment for example.

The composition comprises amino acids that are typically provided for oral consumption and not topical use as in the present composition. The amino acids in the composition have not been previously combined and administered via a topical delivery route. The amino acids may comprise arginine and glutamine in combination.

The composition comprises as active ingredients a combination of amino acids for topical application. The composition comprises L-Arginine and L-Glutamine in combination. L-Arginine increases blood flow and oxygen to the affected skin and acts as an antioxidant, as well as boosts collagen formation in the skin and reduces inflammation. L-Glutamine stimulates collagen production, regulates nitrogen metabolisms and supports the immune system. The L-Arginine and L-Glutamine may be provided to the composition in the same or different amounts by weight.

The active ingredients combination further comprises niacinamide, bromelain, arnica, hyaluronic acid, turmeric, and vitamin C.

In one or more embodiments, active ingredients comprise a combination of amino acids and anti-inflammatory components as described herein. In one or more embodiments the composition having active ingredients including the further comprises additional active and inactive ingredients, including but not limited to:

water, which may be deionized water;

one or more skin protectants, examples of which include dimethicone, zinc oxide, silicone, and combinations thereof;

one or more moisturizing agents, examples of which include but are not limited to glycerin, mineral oil, Cocos nucifera (Coconut) Fruit Extract, and combinations thereof;

one or more emulsifiers and/or stabilizers examples of which include but are not limited to cetearyl alcohol;

thickeners or stabilizers such as xanthan gum;

one or more emollients, examples of which include but are not limited to stearic acid, cetyl alcohol, and combinations thereof;

one or more probiotics such as Lactobacillus;

one or more preservatives and/or natural preservatives such as leuconostoc/radish root ferment filtrate, potassium sorbate, phenoxyethanol, and combinations thereof;

one or more vitamins including ascorbic acid (Vitamin C) for tissue repair and the formation of collagen, niacinamide (Vitamin B-3) for building proteins and protection against environmental damage, hydration and anti-inflammatory properties, tocopherol (Vitamin E), and combinations thereof;

one or more anti-aging components including, but not limited to Rosa canina (rose hip) seed oil;

one or more anti-inflammatory components, examples of which include but not limited to arnica and bromelain;

Simmondsia chinensis (Jojoba) seed oil;

soluble collagen;

one or more antioxidants, examples of which include but are not limited to Helianthus annuus (sunflower) seed oil, tetrahydrocurcumin, and combinations thereof;

Arnica montana (Arnica Flower) extract;

sodium hyaluronate;

methylcobalamin; and combinations thereof.

One example embodiment includes a cosmetic composition comprising deionized water, Simmondsia chinensis (jojoba) seed oil, cetearyl alcohol, stearic acid, cetyl alcohol, Lactobacillus, leuconostoc/radish root ferment filtrate, ascorbic acid, Rosa canina (rose hip) seed oil, soluble collagen, xanthan gum, Arnica montana (Arnica flower) extract, Cocos nucifera (coconut) fruit extract, Helianthus annuus (sunflower) seed oil, l-arginine, l-glutamine, tocopherol, potassium sorbate, bromelain, zinc oxide, niacinamide, maltodextrin, sodium hyaluronate, tetrahydrocurcumin, phenoxyethanol, tetrahydrobisdemethoxydiferuloylmethane, tetrahydrodemethoxydiferuloylmethane, Sodium Benzoate, Sodium Metabisulfite, mineral oil, Methylcobalamin, dimethicone, and glycerin.

For example, amounts (by total weight percentage of the composition) may range as follows: dimethicone may be present in amounts ranging from about 0.1% to about 30%, mineral oil: about 1% to about 20% or more; ascorbic acid: about 0.01% to about 15%; niacinamide about 0.0001% to about 30%, or in the range of 0.0001% to about 20%; soluble collagen: about 0.00005% to about 10%; L-Arginine about 0.1% to about 25%; L-Glutamine: about 0.002% to about 20%; Arnica montana (Arnica Flower) extract: about 0.1 to about 25%; bromelain: about 0.012 to about 1%; sodium hyaluronate: about 0.01% to about 0.05%; Tetrahydrocurcumin: about 0.018% to about 1%; methylcobalamin: about 0.0005% to about 0.5%; Tetrahydrobisdemethoxydiferuloylmethane: about 0.0004% to about 0.5%; Tetrahydrodemethoxydiferuloylmethane: about 0.000002% to about 0.5%, wherein the remaining components include one or more non-active ingredients such as deionized water.

In one embodiment, the cosmetic composition comprises, L-Arginine in a concentration of about 0.01% by weight to about 1% by weight and L-Glutamine in a concentration of about 0.01% by weight to about 1% by weight. The composition of this embodiment further comprises bromelain in a concentration of about 0.01% by weight to about 1% by weight as well as Tetrahydrobisdemethoxydiferuloylmethane in a concentration of about 0.01% by weight to about 1% by weight and Tetrahydrocurcumin in a concentration of about 0.01% by weight to about 1% by weight.

The cosmetic composition according to one or more embodiments described herein can be topically applied to affected skin. What is meant by “affected skin” as used throughout this disclosure is closed skin that may be scarred, injured, damaged, or wrinkled. The composition may also be used topically to aid in the prevention of wrinkles and visual signs of aging. The composition includes a combination of components and one or more of the components have been clinically proven beneficial in the treatment of affected skin.

The compositions described herein cater to all skin types and tones, including the skin of both men and women. The compositions described herein are beneficial to scarred or blemished skin, including but not limited to, skin affected by plastic surgery, blemishes and/or scars due to injury, acne, psoriasis, eczema, and other skin conditions. The compositions have efficacy in fields including plastic surgery, dermatology clinics, and over-the-counter cosmetic and cosmeceutical retailers.

The compositions described herein may comprise an emulsion provided in a form for topical application to the skin for reducing the appearance of scars.

Many of the components of the compositions described herein are widely used for various benefits by way of oral ingestion. Combinations of amino-acids and/or additional components such as anti-inflammatories and essential vitamins has not been indicated for topical use. Topical supplementation according to one or more embodiments described herein provides various technical benefits to a user in addition to the avoidance of daily pill ingestion while remaining highly effective. The user is also able to target areas for use via topical application.

Although the present disclosure has been described with reference to preferred embodiments, workers skilled in the art will recognize that changes may be made in form and detail without departing from the spirit and scope of the disclosure.

1. A cosmetic composition for topical application for reducing the appearance of scars, the composition comprising:
a combination of arginine and glutamine;
one or more skin protectants;
one or more moisturizing agents;
one or more anti-inflammatories;
one or more emulsifiers;
one or more emollients;
one or more probiotics;
a natural preservative;
vitamin c, vitamin B-3, vitamin E or a combination thereof; and
silicone.

2. The cosmetic composition of claim 1 comprising L-Arginine in a concentration of about 0.01% by weight to about 25% by weight and L-Glutamine in a concentration of about 0.002% by weight to about 20% by weight.

3. The cosmetic composition of claim 2 wherein the L-Arginine is present in a concentration of about 0.01% by weight to about 1% and wherein L-Glutamine is present in a concentration of about 0.01% by weight to about 1% by weight.

4. A method of reducing the appearance of a scar on affected skin, the method comprising:
topically applying a cosmetic composition to the affected skin, wherein the cosmetic composition comprises:
a combination of arginine and glutamine;
one or more skin protectants;
one or more anti-inflammatories;
one or more moisturizing agents;
one or more emulsifiers;
one or more emollients;
one or more probiotics;
a natural preservative;
vitamin c, vitamin B-3, vitamin E or a combination thereof; and
silicon.

5. The method of claim 4 wherein the composition comprises L-Arginine in a concentration of about 0.01% by weight to about 25% by weight and L-Glutamine in a concentration of about 0.01% by weight to about 20% by weight.

6. The method of claim 4 wherein the composition further comprises bromelain, tetrahydrobisdemethoxydiferuloylmethane, and tetrahydrocurcumin.

7. An active composition for use in a cosmetic composition for topical application, the active composition comprising a combination of at least two amino acids, niacinamide, bromelain, arnica, hyaluronic acid, turmeric, and vitamin C.

8. The active composition of claim 7 wherein the at least two amino acids comprise arginine and glutamine.
